(19) United States
(12) Patent Application Publication
(10) Pub. No.: US 2026/0102183 A1
(43) Pub. Date: Apr. 16, 2026

(54) SENSOR-INTEGRATED TROCAR FOR LAPAROSCOPIC SIMULATION TRAINING

(71) Applicant: Makam RAMESH, Bengaluru (IN)

(72) Inventors: Makam RAMESH, Bengaluru (IN); Santhosh S KUMAR, Bengaluru (IN); Tulip ALPHONSO, Bengaluru (IN)

(21) Appl. No.: 19/358,072

(22) Filed: Oct. 14, 2025

(30) Foreign Application Priority Data
Oct. 14, 2024 (IN) 202441077883

(51) Int. Cl. A61B 17/34; A61B 17/00; A61B 34/00; A61B 34/10; A61B 90/00

(52) U.S. Cl. CPC A61B 17/34; A61B 17/00234; A61B 34/10; A61B 34/25; A61B 34/76; A61B 90/06; A61B 2017/00707; A61B 2034/101; A61B 2034/254; A61B 2090/062

(57) ABSTRACT

The present subject matter discloses a system for performing simulation training of laparoscopic procedures on a simulation box. The system comprises a plurality of trocar and a laparoscopic hand instrument, with sensors integrated into each of the plurality of trocar of the laparoscopic equipment to monitor critical parameters during training. A processor analyzes the data from the plurality of sensors and provides real-time feedback to the user on the laparoscopic hand instrument's alignment, orientation, and depth. This feedback enables trainees to improve their technique in a controlled and risk-free environment while also supporting precise measurement and monitoring, making it an effective tool for developing laparoscopic surgical skills.

### RELATED APPLICATION

The present application claims priority Indian Patent Application No. 202441077883, filed Oct. 14, 2024, the entire contents of which are hereby incorporated by this reference.

### TECHNICAL FIELD

The present subject matter relates, in general, to training simulators, and particularly but not exclusively, to a laparoscopic simulator for training medical professionals in minimally invasive surgical procedures.

### BACKGROUND

Laparoscopy is a surgical procedure used to examine and operate on organs inside an abdomen. It involves small incisions through which a laparoscope—a thin, lighted tube with a camera—is inserted to provide a visual guide for the surgery. Instruments used in laparoscopy include the laparoscope, trocar, graspers, monitor, forceps, scissors, and more. A crucial instrument is a trocar, a sharp-pointed instrument used in laparoscopy to create access ports through abdominal wall. It allows the insertion of the laparoscope and other surgical tools, facilitating minimally invasive procedures.

### BRIEF DESCRIPTION OF DRAWINGS

The detailed description is described with reference to the accompanying figures. In the figures, the left-most digit(s) of a reference number identify the figure in which the reference number first appears. The same numbers are used throughout the drawings to reference like features and components.

FIG. 1 illustrates a network environment for implementing example techniques for simulation training of a laparoscopic procedure, in accordance with an example implementation of the present subject matter;

FIG. 2 illustrates a system for implementing example techniques for simulation training of a laparoscopic procedure, in accordance with an example implementation of the present subject matter;

FIGS. 3A-3B illustrate a cross-sectional view of a trocar, in accordance with an example implementation of the present subject matter;

FIGS. 3C-3D illustrate another cross-sectional view of the trocar, in accordance with another example implementation of the present subject matter;

FIG. 4 illustrates a schematic block diagram of a training simulator for simulation training of the laparoscopic surgery on a simulated replica organ, in accordance with an example implementation of the present subject matter; and

FIG. 5 illustrates a method for performing a simulation training of the laparoscopic surgery on a simulated box 400 in accordance with an example implementation of the present subject matter.

It may be noted that throughout the drawings, identical reference numbers designate similar, but not necessarily identical, elements. The figures are not necessarily to scale, and the size of some parts may be exaggerated to more clearly illustrate the example shown. Moreover, the drawings provide examples and/or implementations consistent with the description; however, the description is not limited to the examples and/or implementations provided in the drawings.

### DETAILED DESCRIPTION

Laparoscopy is a crucial technique in the modern medical field that has changed the way surgical procedures are performed. It provides a minimally invasive approach, which is associated with numerous benefits, including small incisions, reduced pain for the patients, shorter hospital stays, faster recovery time, and a decreased risk of post-operative complications. These advantages make laparoscopy a preferred method for many types of surgeries, ranging from general procedures to more specialized interventions in gynecology, urology, and gastrointestinal surgery. Despite these benefits, the instrument used in the laparoscopic procedures poses several challenges that hinder their overall effectiveness and safety.

The challenges associated with laparoscopic procedures are further hindered by deficiencies in conventional training practices for the surgeons. Mastering the use of laparoscopic equipment requires a delicate balance of precision, control, and tactile awareness, skills that are not easily developed through traditional training methods. While there are existing training simulators and techniques, there is still room for significant improvement and optimization. One ongoing challenge is the accurate assessment of surgical skills that can both provide realistic training and assess the skills of trainees while using the laparoscopic hand instruments, particularly for laparoscopic surgery.

Further, the conventional training simulators often fall short in replicating the nuanced conditions of the actual surgical procedures. These systems may not adequately convey the spatial and mechanical challenges encountered during real operations, making it difficult for trainees to master critical aspects such as instrument positioning, angulation, and coordinated movement. Without this feedback, trainees may struggle to accurately gauge the correct position, required rotation and angle, and overall technique needed to successfully perform the laparoscopic surgery. As a result, conventional simulators are limited in their ability to effectively prepare surgeons for the complexities of real-world procedures, making it challenging to develop the precision and tactile awareness necessary for optimal patient outcomes.

Therefore, there is a need for a system that addresses the above-mentioned limitations of both surgical performance and training efficacy in the laparoscopic training simulators. By incorporating features such as improved ergonomics and realistic simulation of surgical scenarios, the training models could significantly improve the learning experience for trainees while ultimately minimizing risks for patients.

In accordance with the present subject matter, a system for simulation training of a laparoscopic procedure is provided. In example implementations, the system provides to solve the above-mentioned problems.

In accordance with an implementation, the system comprises a plurality of laparoscopic equipment for performing the simulation training on a simulated replica organ. The laparoscopic equipment comprises a plurality of trocars having a funnel-shaped upper portion transitioning into a cylindrical lower portion. Each of the plurality of trocar comprises an elongated hollow cannula, the elongated cannula having a proximal end positioned near the simulated replica organ and a distal end extending away from the simulated replica organ. The trocar further comprises a sleeve configured to be inserted inside the elongated hollow cannula. The sleeve comprising a plurality of grooves that align with an internal hub located within the upper portion of the trocar. The internal hub comprises a first gear profile that corresponds to a second gear profile located within the upper portion of the trocar, such that as the sleeve rotates, the second gear profile on the upper portion of the trocar also rotates.

The laparoscopic equipment further comprises a laparoscopic hand instrument, the laparoscopic hand instrument comprises an elongated shaft configured to move telescopically and rotate in unison with the sleeve within the hollow cannula of the trocar and be insertable into the internal cavity of the simulated replica organ. The elongated shaft of the laparoscopic hand instrument comprises a proximal end and a distal end, with the proximal end extending into an internal cavity of the simulated replica organ and being configured to accommodate one or more surgical instruments. The upper portion of the trocar houses a plurality of sensors.

The plurality of sensors includes a first sensor configured to measure a spatial orientation and coordinates of the laparoscopic hand instrument during the simulation training of the laparoscopic procedure, and a second sensor configured to measure depth of insertion of the laparoscopic hand instrument during the simulation training of the laparoscopic procedure. Further, the plurality of sensors also includes a third sensor configured to measure rotational movement of the laparoscopic hand instrument during the simulation training of the laparoscopic procedure, and a fourth sensor configured as a homing device to detect presence of the laparoscopic hand instrument during the simulation training of the laparoscopic procedure. The system further comprises a processor operatively coupled to the plurality of sensors to provide feedback during the simulation training of the laparoscopic procedure.

In an example, the simulated replica organ may be designed using organ tissue-like synthetic materials that closely replicate the texture, consistency, and responsiveness of a real human organ. In said example, these materials may be carefully selected to provide appropriate tactile feedback, allowing users to experience a lifelike sensation when manipulating laparoscopic instruments during simulation training. The simulator accurately mimics the resistance, elasticity, and deformation characteristics of actual organ tissues, ensuring that trainees develop a realistic sense of instrument handling and surgical technique. This realistic feedback mechanism enables precise hand-eye coordination, depth perception, and force application, essential for mastering laparoscopic surgery.

By integrating the above-mentioned plurality of sensors and by analyzing the data gathered through such, the present system enhances the accuracy, realism, and efficiency of laparoscopic training simulator by mimicking an environment for the trainees that closely mirrors real surgery and further by providing comprehensive data on spatial orientation, depth of insertion, rotational movement, and the presence of the laparoscopic hand instrument in real-time. These tracking capabilities enable detailed analysis of user's performance, providing actionable insights to refine skills and improve procedural outcomes. Further, by incorporating high-fidelity materials in the simulated replica organ, the system enhances the realism of the training experience, allowing trainees to develop accurate surgical techniques, improve hand-eye coordination, and gain confidence in performing laparoscopic procedures in a controlled, risk-free environment.

The above-mentioned implementations are further described herein with reference to the accompanying figures. It should be noted that the description and figures relate to exemplary implementations and should not be construed as a limitation to the present subject matter. It is also to be understood that various arrangements may be devised that, although not explicitly described or shown herein, embody the principles of the present subject matter. Moreover, all statements herein reciting principles, aspects, and embodiments of the present subject matter, as well as specific examples, are intended to encompass equivalents thereof.

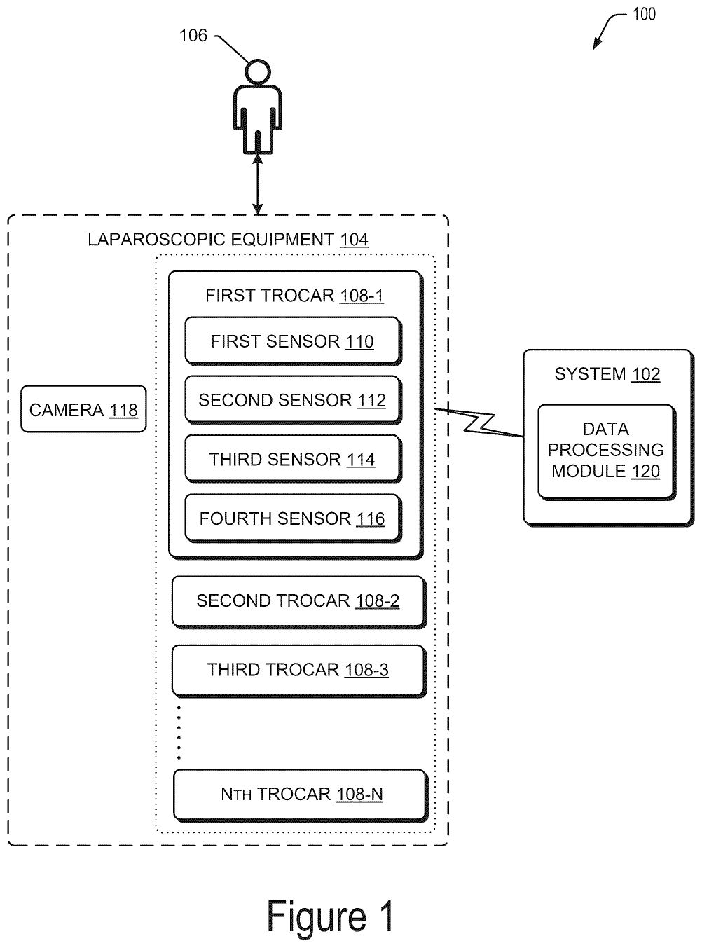

FIG. 1 illustrates an environment 100 depicting systems for simulation training of a laparoscopic procedure, in accordance with an example implementation of the present subject matter.

Training simulators for laparoscopy procedures are essential due to the unique challenges posed by this surgical technique. Laparoscopy, with its minimally invasive approach, offers significant benefits such as reduced patient pain, smaller incisions, shorter hospital stays, faster recovery, and a lower risk of post-operative complications. However, these advantages are contingent on the surgeon's ability to master the precise and controlled use of laparoscopic instruments. This critical skill is difficult to develop through traditional training simulators, which often do not provide tactile feedback and lack the complexity and realism of actual surgical scenarios.

In accordance with an example of the present subject matter, network environment 100 comprises a system 102 implemented to provide feedback to the user training on the laparoscopic training simulator.

In an example, the system 102 may be any computing device, such as a server, a desktop computer, a laptop, a smartphone, or a tablet. The system 102 may comprise one or more processors for executing the process for simulation training of a laparoscopic procedure. In an example, the processor may be implemented as microprocessors, microcomputers, microcontrollers, digital signal processors, central processing units, state machines, logic circuitries, and/or any devices that manipulate signals based on operational instructions. The system 102 may comprise a memory (not shown) for storing the instructions executable by the one or more processors. The memory may include any computer-readable medium known in the art including, for example, volatile memory (e.g., RAM), and/or non-volatile memory (e.g., EPROM, flash memory, etc.). The memory may also be an external memory unit, such as a flash drive, a compact disk drive, an external hard disk drive, or the like.

In an example, the system 102 is operatively coupled to a plurality of laparoscopic equipment(s) 104 for performing the simulation training on a simulated replica organ by a user 106. In an example, the plurality of laparoscopic equipment comprises a plurality of trocar 108-1, 108-2, . . . , 108-N. Each of the plurality of trocar 108-1, 108-2, . . . , 108-N have a funnel-shaped upper portion transitioning into a cylindrical lower portion, and comprises an elongated hollow cannula, the elongated cannula having a proximal end positioned near the simulated replica organ and a distal end extending away from the simulated replica organ. In an example, each of the plurality of trocar 108-1, 108-2, . . . , 108-N may also comprise an obturator (not shown) insertable into the cannula and having a sharp dilating tip to pierce a tissue boundary of the simulated replica organ.

In an example, each of the plurality of trocar 108-1, 108-2, . . . , 108-N comprises an elongated hollow cannula configured to be inserted into the top surface of the simulated replica organ. The elongated cannula having a proximal end and a distal end, with distal end to remain outside the simulated replica organ and proximal end being configured to be inserted inside the trocar such that it engages or interfaces with the simulated replica organ. In an example, the internal hub comprises a first gear profile that corresponds to a second gear profile located within the upper portion of the trocar, such that as the sleeve rotates, the second gear profile also rotates.

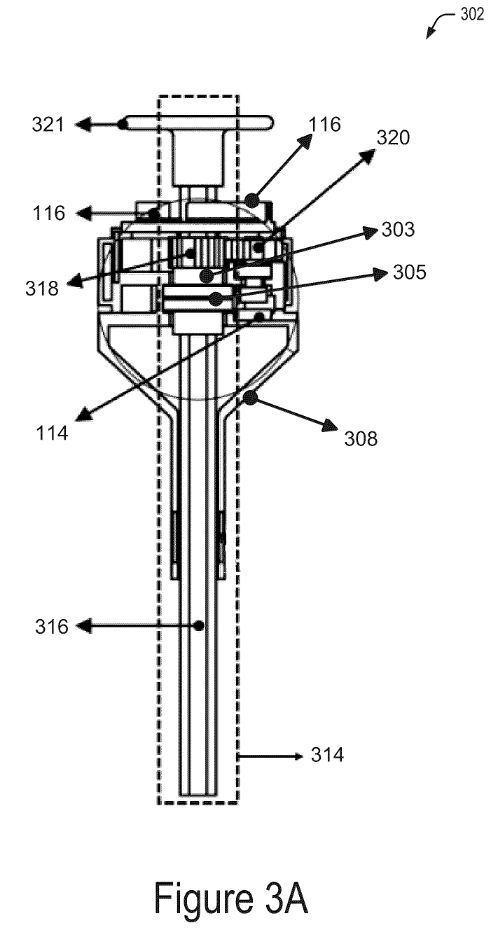

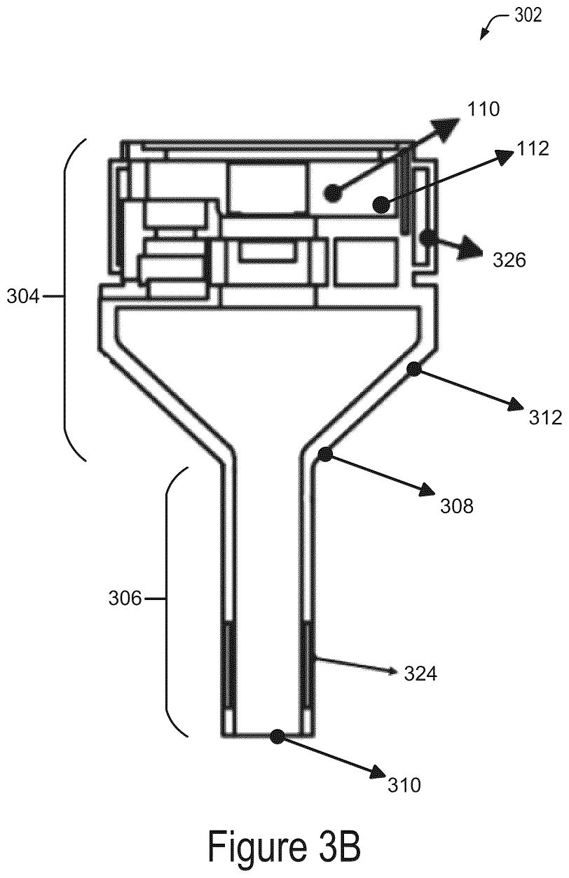

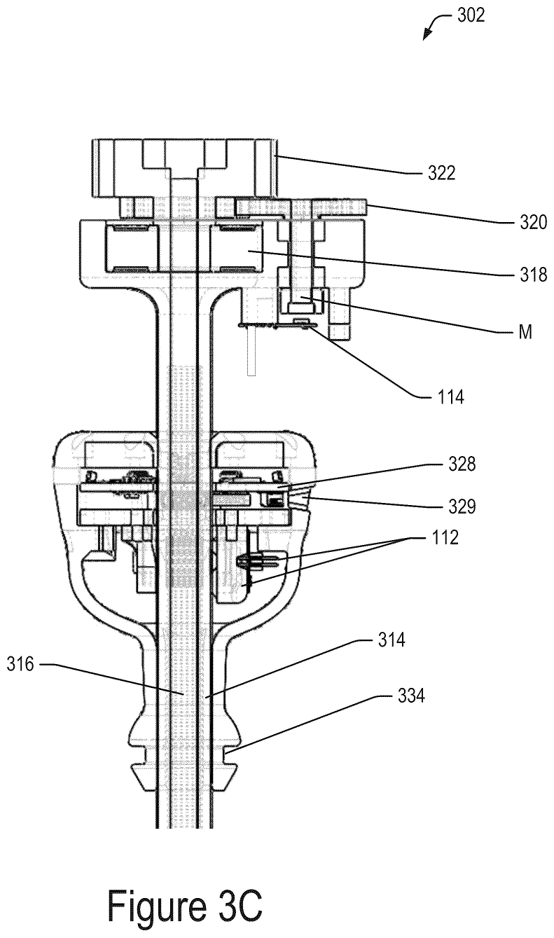

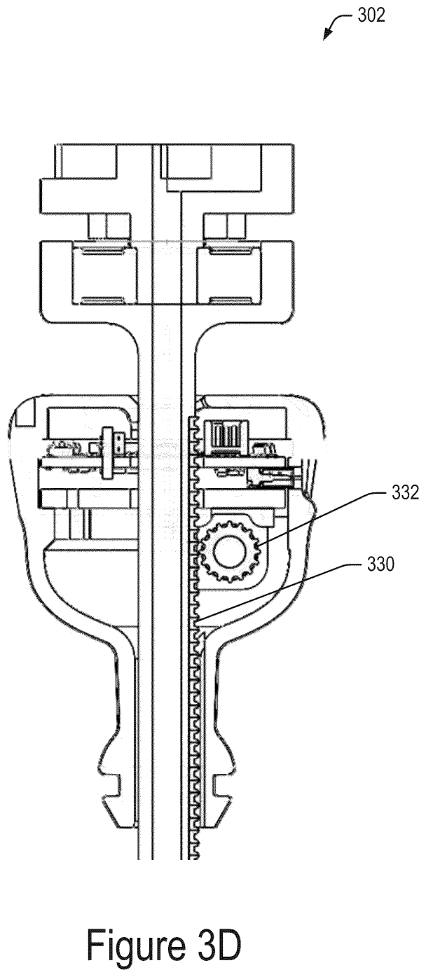

In one example, the sleeve and its corresponding grooves, aligns with the internal hub (as illustrated in FIG. 3A) within the upper portion of each of the plurality of trocar 108-1, 108-2, . . . , 108-N. In an example, the internal hub may have a hexagonal shape. However, the design is not limited to this configuration and may adopt other shapes as well, such as triangle, square, rectangle, or any other polygonal shape, depending on the specific requirements of the application.

In an example, the laparoscopic equipment(s) 104 further comprises a laparoscopic hand instrument which comprises an elongated shaft configured to move telescopically and rotate in unison with the sleeve within the hollow cannula of each of the plurality of trocar 108-1, 108-2, . . . , 108-N and insertable into the internal cavity of the simulated replica organ. In an example, the laparoscopic hand instrument may include but not limited to instruments like graspers, scissors, needle holders, bipolar forceps, suction-irrigation devices, etc.

In an example, the upper portion of each of the plurality of trocar 108-1, 108-2, . . . , 108-N comprises a plurality of slots to house a plurality of sensors. In an example, the plurality of sensors include a first sensor 110 configured to measure a spatial orientation and coordinates of the laparoscopic hand instrument during the simulation training of the laparoscopic procedure by the user 106. Further, the plurality of sensors includes a second sensor 112 configured to measure depth of insertion of the laparoscopic hand instrument during the simulation training of the laparoscopic procedure. Further, the plurality of sensors may include a third sensor 114 configured to measure rotational movement of the laparoscopic hand instrument during the simulation training of the laparoscopic procedure by the user 106. Further, the plurality of sensors may include a fourth sensor 116 configured as a homing device to detect presence of the laparoscopic hand instrument during the simulation training of the laparoscopic procedure by the user 106.

In an example implementation of the present subject matter, the laparoscopic equipment(s) 104 may also comprise one or more image capturing device 118, herein as ‘camera’. In an example, the camera 118 may be placed within the laparoscopic equipment 104 to simulate its real-world application. For instance, the camera 118 may be installed at the proximal end of the laparoscope hand instrument and positioned to be inserted through the trocar 108 during the simulation, replicating its placement in actual laparoscopic procedures. Alternatively, it may be mounted within the simulated abdominal cavity to provide a fixed perspective of the surgical site. The camera's 118 placement ensures it captures clear, high-resolution visuals of the simulated surgical environment, emulating the field of view experienced during real laparoscopic surgeries.

In an example, the camera 118 is integrated into the laparoscopic equipment 104 to capture real-time, high-resolution images or videos of a simulated surgical environment when performed by the user 106. In an example, this visual data is then displayed on a user interface, allowing users 106 to observe their surgical techniques being performed on the simulated replica organ during the laparoscopy simulation training.

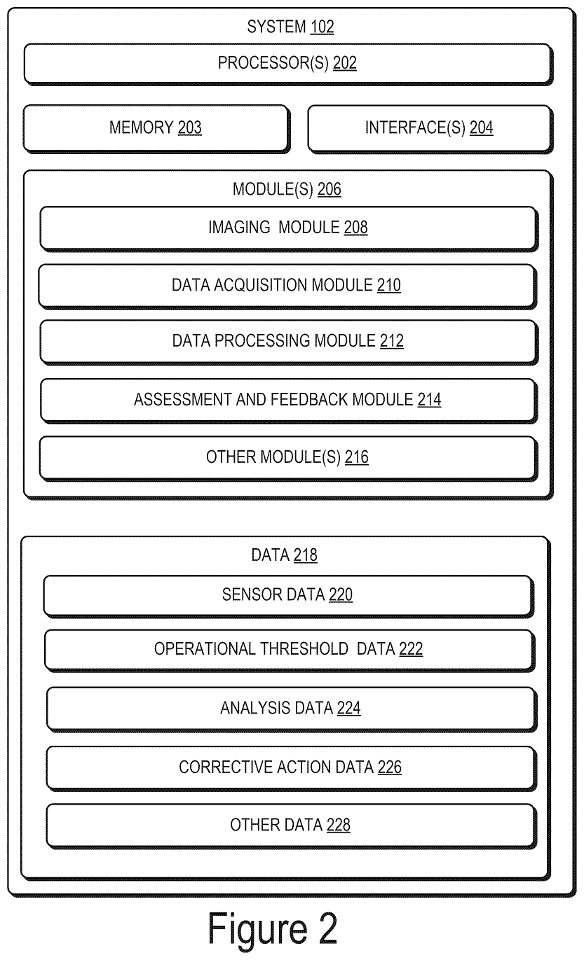

In an example implementation of the present subject matter, the system 102 may comprise a data processing module 120 that is configured to process the data received from the plurality of sensors installed on the trocar 108 of the laparoscopic equipment(s) 104. In an example, the system 102 is further configured to provide the user 106 a feedback based on the collected sensor data, regarding critical parameters such as alignment of the laparoscopic instrument, orientation of the laparoscopic instrument, and the depth at which the laparoscopic instrument is inserted by the user 106 during the simulated laparoscopic procedure. In an example, the feedback may be displayed on a user interface in the form of visual indicators, numerical metrics, or graphical representations, allowing the user to make precise adjustments as needed. This real-time feedback ensures that the user can refine their technique, improve accuracy, and develop the necessary skills for performing safe and effective laparoscopic surgeries. For an explanation of the implementation and operation of the system 102 to perform the feedback to the user 106, reference is made to FIG. 2.

FIG. 2 provides a system for implementing simulation training of a laparoscopic procedure, providing feedback to a trainee 106 using a training simulator, in accordance with an example implementation of the present subject matter.

As depicted in FIG. 2, in an example implementation, the system 102 may include a processor 202 and a memory 203 coupled to the processor 202. In an example, the processor 202 may be implemented as microprocessors, microcomputers, microcontrollers, digital signal processors, central processing units, state machines, logic circuitries, and/or any devices that manipulate signals based on operational instructions. The memory 203 may include any computer-readable medium known in the art including, for example, volatile memory (e.g., RAM), and/or non-volatile memory (e.g., EPROM, flash memory, etc.). The memory 203 may also be an external memory unit, such as a flash drive, a compact disk drive, an external hard disk drive, or the like.

As also depicted in FIG. 2, in an example implementation, interface(s) 204 may be coupled to the processor 202. The interface(s) 204 may include a variety of software and hardware interfaces that allow interaction of the user 106 with the system 102 along with other communication and computing devices. Further, in an example, the interface(s) 204 may couple a user device 106 to the system 102. In an example, the interface(s) 204 may also include display capabilities to present camera imaging and real-time video feed to the users 106 while performing the simulation training. This real-time visual feedback may allow users 106 to observe and analyze their techniques during the simulated laparoscopic procedures, enhancing the learning experience and enabling more effective skill development.

In an example, the system 102 may also comprise module(s) 206 and data 218 coupled to the processor 202. In one example, the module(s) 206 and data 218 may reside in memory 204.

In an example, the data 218 may comprise sensor data 220, an operational threshold data 222, analysis data 224, corrective action data 226, and other data 228. The module(s) 208 may include routines, programs, objects, components, data structures, and the like, which perform particular tasks or implement particular abstract data types. The module(s) 208 may further include modules that supplement applications on the system 102 for laparoscopy simulation, for example, modules of an operating system. The data 218 serves, amongst other things, as a repository for storing data that may be fetched, processed, received, or generated by one or more of the module(s) 206. The module(s) 206 may include an image processing module 208, a data acquisition module 210, a data processing module 212, an assessment and feedback module 214, and other module(s) 216. The other module(s) 216 may include programs or coded instructions that supplement applications and functions, for example, programs in the operating system of the system 102.

As explained previously, each of the trocar among the plurality of trocar 108-1, 108-2, . . . , 108-N of the laparoscopic equipment(s) 104 comprises a plurality of sensors comprising, but not limited to, a first sensor 110, a second sensor 112, a third sensor 114, and a fourth sensor 116. In an example, these plurality of sensors are strategically positioned on the upper portion of the trocar 108 to monitor various parameters crucial for providing accurate feedback during simulation training of the laparoscopy procedure. The precise location of each of the plurality of sensors on the trocar 108 of the laparoscopic equipment(s) 104 has been explained in detail later in FIG. 3.

In an example, the first sensor 110 is an inertial measurement unit (IMU) integrated on the trocar to collect data or readings pertaining to angular velocity, acceleration, and movement of the laparoscopic hand instrument as it interacts with the simulated replica organ. In an example, this data from the first sensor 110 may be referred to as ‘first sensor data’. In an example, the first sensor ensures precise monitoring of the laparoscopic hand instrument's motion, capturing even subtle in trajectory, speed, or stability. In an example, the first sensor 110 works in conjunction with other sensors to provide feedback to the user 106 operating on the simulated replica organ. This feedback includes critical data on the usage of the laparoscopic hand instrument, such as depth of insertion, spatial orientation, and movement dynamics, enabling more precise control and enhancing the overall training experience.

In an example implementation of the present subject matter, the trocar 108 further comprises a second sensor 112. In an example, the second sensor 112 is a Time-of-Flight (ToF) sensor to be used for measuring the data pertaining to the depth (z-axis movement) of insertion of laparoscopic hand instrument by the user 106. In an example, the second sensor may use an infrared light to calculate the distance between the proximal end of the laparoscopic hand instrument and the targeted tissue boundary target of the simulated replica organ, ensuring precise insertion depth during the procedure in simulation training by the user 106. In another example, the second sensor 112 may incorporate a rack and pinion mechanism. As the laparoscopic hand instrument is moved in or out, the linear movement of the rack drives the rotation of the pinion, thereby enabling detection of the instrument's displacement. This arrangement is illustrated and described in detail with reference to FIGS. 3C and 3D. In an example, the second sensor 112 may be at least one of a time-of-flight (ToF) sensor, linear variable differential transformer (LVDT) sensor or encoder-based sensor, such as, but not limited to magnetic rotary encoder, positioned at a specified location along upper portion of the trocar.

In an example, the data from the second sensor 112 may be referred to as ‘second sensor data’. In an example, the second sensor 112 works in conjunction with other sensors to provide feedback to the user 106 operating on the simulated replica organ. This feedback includes critical data on the usage of the laparoscopic hand instrument, such as depth of insertion, spatial orientation, and movement dynamics, enabling more precise feedback and enhancing the overall training experience.

In an example implementation of the present subject matter, the trocar 108 further comprises a third sensor 114. In an example, the third sensor 114 may be a magnetic encoder that measures the rotational movement of the laparoscopic hand instrument during the simulation training of the laparoscopic procedure. In an example, the third sensor 114 works by utilizing a gear shaft (as shown in FIG. 3) that aligns with an internal hub of the trocar (also shown in FIG. 3). In an example, this internal hub features a specific gear profile. As the laparoscopic hand instrument rotates within the trocar 108, the gear shaft of the third sensor 114 rotates correspondingly. This mechanical interaction allows the third sensor 114 to convert the rotational motion of the laparoscopic hand instrument into quantifiable data. In an example, the data from the third sensor 114 may be referred to as ‘third sensor data’.

In an example, the third sensor 114 works in conjunction with other sensors to provide feedback to the user 106 operating on the simulated replica organ. This feedback includes critical data on the usage of the laparoscopic hand instrument, such as depth of insertion, spatial orientation, and movement dynamics, enabling more control and enhancing the overall training experience.

In an example implementation of the present subject matter, the trocar 108 further comprises a fourth sensor 116. In an example, the fourth sensor may be a plurality of proximity sensors, laser sensors, hall effect sensors, etc., embedded around each of the trocar among the plurality of trocar 108-1, 108-2, . . . , 108-N to serve as a homing device. The fourth sensor is configured to calculate data pertaining to the detection of the presence of the laparoscopic hand instrument as it approaches the trocar and to ensure precise alignment before insertion by the trainee. In an example, the data from the fourth sensor may be referred to as ‘fourth sensor data.’

In an example, the fourth sensor 116 works in conjunction with other sensors to provide feedback to the user 106 operating on the simulated replica organ. This feedback includes critical data on the usage of the laparoscopic hand instrument, such as depth of insertion, spatial orientation, and movement dynamics, enabling more control and enhancing the overall training experience. By detecting the presence of the laparoscopic hand instrument, the fourth sensor ensures proper alignment, minimizing the risk of misalignment during instrument insertion and enhancing the overall accuracy of the simulation training process for the user 106.

In an example, the system 102 comprises an imaging module 208 configured to receive real time videos and images from the camera 118. In an example, the imaging module 208 plays a crucial role in processing and displaying the visual data being captured during the simulated laparoscopic procedures in real time. In an example, the imaging module 208 is to handle high-resolution video streams and images in real-time, providing visual feedback to the trainees 106 to accurately assess tissue structures, identify anatomical landmarks, and precisely manipulate the laparoscopic hand instrument in the simulated replica organ within the simulated environment.

In an example, to enhance the image capturing process during the simulation training of the user 106, the imaging module 208 may comprise several control features. In an example, the imaging module 208 may include a focus control feature that allows for the adjustment of the camera's focus when it is too close to the location at which the user 106 is performing the surgery, which may help ensure clear visualization of the surgical site. Additionally, the imaging module 208 may also include a position control that allows the camera's orientation to be adjusted, enabling it to capture images of the surgery from different angles as needed during the simulation training. Furthermore, the imaging module 208 may include a light brightness control that adjusts the illumination of the captured videos or images to ensure proper lighting, either by increasing or decreasing brightness. In an example, these controls in the imaging module 208 may provide users 106 with a more dynamic and interactive training experience, potentially helping them refine their laparoscopic skills with realistic visual feedback.

In an example, the system 102 comprises a data acquisition module 210 configured to receive first sensor data, second sensor data, third sensor data, and fourth sensor data from among the plurality of sensors, herein referred to as ‘sensor data 220’, each serving a specific function in monitoring the movement of the laparoscopic hand instrument during simulation training. In an example, the sensor data 220 may include the first sensor dataset collected from the first sensor 110, providing information related to the instrument's angular velocity, linear acceleration, and movement within the simulated environment. In an example, the sensor data 220 may further comprise the second sensor dataset collected from the second sensor 112, the third sensor dataset collected from the third sensor 114, the fourth sensor dataset collected from the fourth sensor 116.

In an example, the data acquisition module 210 is configured to collect the sensor data 220 from the plurality of sensors. In an example, the sensor data 220 includes information from all the sensors integrated into the trocar, such as the first sensor 110, second sensor 112, third sensor 114, and fourth sensor 116. This data is then stored in memory 203 for further processing and analysis. In an example, the data acquisition module 210 ensures that all relevant sensor inputs of the sensors installed on each of the plurality of trocar 108-1, 108-2, . . . , 108-N are collected in real-time, allowing for accurate tracking and storage of performance metrics during the simulation training of the user.

In an example, the system 102 comprises a data processing module 212 for processing the sensor data 220 collected by the data acquisition module 210. In an example, the data processing module 212 is similar to the data processing module 120 as illustrated in FIG. 1. In an example, the data processing module 212 is designed to analyze and interpret the raw sensor data 220. In an example, the analysis by the data processing module 212 may comprise receiving sensor data 220 from the plurality of sensors integrated within the trocar 108 of the laparoscopic equipment(s) 104 including the first sensor 110, the second sensor 112, the third sensor 114, and the fourth sensor 116. Further, the data processing module 212 comprises comparing the received sensor data 220 against a predefined operational threshold associated with each of the plurality of sensors to detect deviations.

In an example, the operational thresholds associated with each of the plurality of sensors may be set by experts in the field of laparoscopic surgery. These thresholds may represent optimal ranges or limits for various parameters such as instrument alignment, orientation, depth of insertion, and rotational movement. By utilizing expert-defined thresholds, the system 102 may provide feedback that aligns with best practices in laparoscopic procedures, potentially enhancing the quality and effectiveness of the simulation training.

In an example, the data processing module 212 further comprises transmitting the processed data, herein referred to as ‘analysis data 224’ to the user interface 204 to provide real-time feedback to the user 106 comprising deviation value with respect to the operation threshold values on laparoscopic instrument alignment, orientation, and depth during the simulation training of the laparoscopic procedure by the user 106.

In an example, the system 102 may further comprise an assessment and feedback module 214 for providing trainees 106 with feedback and suggestions on various aspects of their laparoscopic simulation training to improve their performance. In an example, the assessment and feedback module 214 may analyze the processed data 220 from the data processing module 212 and generate comprehensive feedback and suggestions for the trainees 106 by utilizing a corrective action data 226.

In an example, the corrective action data 226 may comprise a comprehensive repository of information related to proper laparoscopic techniques, best practices, and recommended actions for various scenarios encountered during laparoscopic procedures. In an example, this repository may also include photos or videos demonstrating the correct execution of specific techniques, providing visual guidance for trainees 106. In an example, these visual aids may illustrate proper instrument handling, optimal trocar placement, or effective tissue manipulation techniques.

In an example, this repository of the corrective action data 226 may be prepared and curated by experienced medical professionals, ensuring the accuracy and relevance of the information. If a trainee 106 performs a technique incorrectly during the simulation, the system 102 may access this repository to display the correct method. This feature may allow trainees to compare their performance with expert demonstrations, potentially facilitating more effective learning and skill development. In an example, the system 102 may present side-by-side comparisons or overlay the correct technique onto the trainee's actions, highlighting areas for improvement and reinforcing proper procedural methods.

In an example, the corrective action data 226 may be continuously updated based on new research, expert input, and aggregated performance data from multiple training sessions. This dynamic nature of the corrective action data 226 may allow the assessment and feedback module 214 to provide up-to-date and relevant guidance, potentially adapting to evolving best practices in laparoscopic surgery.

In an example implementation, the assessment and feedback module 214 may use the corrective action data 226 to create personalized improvement plans for trainees. By comparing the trainee's performance metrics against the corrective action data 226, the module may identify specific areas for improvement and suggest a tailored set of exercises or practice scenarios to address these areas.

Thus, by integrating the above-mentioned plurality of sensors, the system significantly enhances the accuracy, realism, and efficiency of the laparoscopic training simulator. These sensors provide detailed data on spatial orientation, depth of insertion, rotational movements, and the presence of the laparoscopic hand instrument during the use, enabling advanced tracking and analysis of the user's performance. This comprehensive feedback offers actionable insights to refine surgical skills and improve procedural outcomes, while also addressing the limitations of existing laparoscopic training simulators.

FIG. 3A-3B illustrate a cross-sectional view of a trocar 302 for performing the simulation training by a user 106 on a simulated replica organ (not shown), and FIG. 3C-3D illustrate another cross-sectional view of the trocar 302, in accordance with another example implementation of the present subject matter. FIGS. 3A-3D are explained together to provide a comprehensive description of the laparoscopic equipment 104 of the present training simulator.

In an example, the laparoscopic equipment 104 comprises a trocar 302 and a laparoscopic hand instrument (not shown). In an example, the trocar 302 have a funnel-shaped upper portion 304 transitioning into a cylindrical lower portion 306. In an example, each of the trocar among the plurality of trocar 108-1, 108-2, . . . , 108-N is similar to the trocar 302. In an example, the trocar 302 includes an elongated hollow cannula 308 having a proximal end 310 positioned near the simulated replica organ and a distal end 312 positioned away from the simulated replica organ, with proximal end being configured to enter inside the trocar such that it engages or interfaces with the simulated replica organ

In an example, the trocar 302 also comprises a sleeve 314 having a hollow structure 316 that extends into the internal cavity of the simulated replica organ and configured to be inserted inside the elongated hollow cannula 308, allowing for the guided movement of the laparoscopic hand instrument. In an example, the sleeve 314 is securely fixed to the trocar 302, allowing only rotational movement while restricting any in-and-out translation along with the laparoscopic hand instrument. Additionally, during operation the sleeve 314 may be configured to attach to the laparoscopic hand instrument, ensuring a stable and controlled motion. This secure attachment enhances precision, providing the user 106 with improved handling and maneuverability of the instrument throughout the procedure. In an example, the sleeve 314 features a hole (not shown) at its top, through which the laparoscopic hand instrument is inserted to reach the internal cavity of the simulated replica organ. This hole ensures proper alignment and facilitates the smooth movement of the hand instrument by the user 106 during the simulation training.

In an example, the sleeve 314 also functions to guide the laparoscopic hand instrument as it moves telescopically within the cannula, enabling control and rotation in unison with the instrument. In an example, the sleeve comprises a plurality of grooves that align with an internal hub 303 (as illustrated in FIG. 3A) located within the upper portion of the trocar 302. In an example, the internal hub 303 comprises a first gear profile 318 that corresponds to a second gear profile 320 located within the upper portion 304 of the trocar 302. In an example, ensure the sleeve 314 remains firmly attached to the laparoscopic hand instrument, a spring-loaded locking mechanism 322 (as illustrated in FIG. 3C) may also be provided. This mechanism provides a secure and rigid connection for rigidly fixing the trocar sleeve onto the laparoscopic instrument, preventing unintended slippage during surgical training or use.

In an example, the laparoscopic hand instrument comprises an elongated shaft (not shown) configured to move telescopically and rotate in unison with the sleeve within the hollow cannula 308 and be inserted into an internal cavity of the simulated replica organ. In one example, the sleeve 314 and its corresponding grooves, which align with the internal hub within the upper portion of each of the plurality of trocar 108-1, 108-2, . . . , 108-N, may have a hexagonal shape. However, the design is not limited to this configuration and may adopt other shapes as well, depending on the specific requirements of the application.

In an example, the internal hub 303 may comprise a circular rotary element 305 (as illustrated in FIG. 3A) designed to regulate and constrain the movement of the sleeve 314 when it is inserted in the trocar 302. The sleeve 314 features the above mentioned first gear profile 318 at one of its ends, which corresponds to the second gear profile 320, ensuring synchronized rotational motion for collecting sensor data pertaining to rotation of the laparoscopic hand instrument. The rotary element 305 is strategically positioned and fixed within the top portion of the trocar 302, allowing for smooth and controlled rotation of the sleeve 314 while minimizing friction. Additionally, the rotary element 305 plays a crucial role in stabilizing the internal hub 303 by providing structural support, ensuring precise alignment, and reducing mechanical wear over prolonged use. This configuration enhances the overall durability and efficiency, enabling seamless interaction between the laparoscopic hand instrument and the trocar 302 during simulation training.

In operation, during simulation training, as the user 106 rotates the laparoscopic hand instrument, the sleeve 314 also rotates correspondingly within the hollow cannula 308 of the trocar 302. This movement engages the first gear profile 318 on the sleeve with the second gear profile 320 located within the upper portion 304 of the trocar. As the laparoscopic hand instrument rotates, the first gear 318 transfers motion to the second gear 320, causing it to rotate. In an example, the rotation of the second gear 320 is measured and converted into quantifiable data, allowing for precise tracking of instrument movement and providing valuable feedback on the user's technique during training.

In an example implementation, the sleeve 314 may feature a hexagonal profile and is securely fixed onto the laparoscopic hand instrument, ensuring proper alignment and stability during simulation training. This design may allow the sleeve 314 to pass smoothly through the internal hub 303, having corresponding hexagonal internal profile, inside the trocar, facilitating control and movement of the instrument. In an example, the sleeve 314 may be removed from the trocar, enabling it to be detached and placed onto another laparoscopic hand instrument with the same profile. This interchangeability enhances the versatility of the system, allowing different surgical tools to be used within the same trocar setup while maintaining consistent alignment and rotational accuracy.

In an example, the upper portion 304 of the trocar 302 comprises a plurality of slots to house the plurality of sensors, as discussed earlier. These may include the first sensor 110, the second sensor 112, the third sensor 114, and the fourth sensor 116. In an example, each of these sensors are strategically placed to capture specific data related to the movement and positioning of the laparoscopic hand instrument during the simulation training. In an example, the first sensor 110 and the second sensor 112 may be positioned at a specified location along the upper portion 304 of the trocar 302. In an example, the third sensor 114 comprises a profile having a second gear 320 configured to align with the internal hub of the trocar 302, which features the gear profile 320 as the laparoscopic hand instrument rotates within the trocar 302 the gear shaft 320 of the third sensor 114 rotates correspondingly converting the rotational motion of the laparoscopic hand instrument into a quantifiable data. In an example, the fourth sensor 116 and the fifth sensor 118 may be positioned at the top of the trocar 302.

In an example, the sleeve 314 may include a disc 321 positioned on its top portion, designed to interact with the second sensor, i.e., a Time-of-Flight (ToF) sensor. During simulation training, the disc 321 obstructs the ToF sensor's light source, causing the emitted light to reflect back to the sensor's receiver. This interaction enables the system 102 to measure the up-and-down movement of the laparoscopic hand instrument.

In another example, as illustrated in FIGS. 3C-3D, the second sensor 112 may be an encoder-based sensor, such as a magnetic rotary encoder, configured to measure the depth of insertion of the laparoscopic hand instrument during simulation training. In the trocar design in accordance with the depicted embodiment, the Z-axis (depth) measurement functionality is integrated into the body of the trocar 302 and is implemented using a rack 330 and pinion 332 mechanism. As illustrated in FIG. 3D, a linear rack 330 may be mounted along one face of a sleeve 314 that securely fits over the laparoscopic instrument. As the instrument is moved longitudinally during the simulation training, either inserted or withdrawn, the rack 330 may undergo linear displacement, which in turn may cause the pinion 332 to rotate. This rotational motion is detected by the second sensor 112 coupled to the pinion 332, and the resulting signal is converted into an accurate depth measurement.

In said embodiment, the sleeve 314 performs a dual function: it acts as the structural support for the rack 330 while also guiding the laparoscopic instrument to ensure stable alignment within the trocar 302. Additionally, at the end of the sleeve opposite the rack 330, the third sensor 114, which may be a magnetic encoder reader, is integrated to capture the rotational motion of the laparoscopic tool. In this configuration, a planetary gear assembly 320 (as mentioned previously as ‘second gear’) may be employed in conjunction with a bearing 318 (as mentioned previously as ‘first gear’) to facilitate smooth and controlled rotational input. In an example, a magnet M may be positioned above the magnetic encoder reader 114 to complete the sensing mechanism. In an example, this rotational movement may also be tracked using a magnetic encoder similar to the second sensor 112, thereby enabling the system 102 to monitor both translational (depth) and rotational motions of the instrument with high precision. This dual-axis sensing capability enhances the fidelity of the simulation by providing real-time feedback on instrument handling.

In an example, the plurality of sensors may have their own dedicated circuit board or may be embedded in a single or multiple printed circuit boards (PCBs) 328. The PCB 328 may be installed on the top portion of the trocar 302, providing a compact and integrated solution for housing the plurality of sensors. In an example, the top portion of the trocar 302 may also include a C-pin socket 329 configured to interface with the PCB 328, thereby facilitating secure electrical connectivity for power and data transmission. This arrangement may allow for efficient use of space within the trocar 302 while ensuring proper connectivity and communication between the sensors and the data acquisition module 210. In an example, utilizing a PCB-based sensor integration may enhance the overall reliability and durability of the sensor system, potentially simplifying maintenance and replacement procedures if needed.

In an example, the distal end of the laparoscopic hand instrument may comprises a handle (not shown) to provide a secure and ergonomic grip for the user 106 while performing simulation training of the laparoscopic procedure. In an example, the handle allows the user 106 to maneuver the laparoscopic hand instrument with precision and control during the simulation training. In an example, the handle may be configured to facilitate various movements, such as rotation, insertion, and retraction, required for performing laparoscopic procedures on the simulated replica organ. In an example, the handle may be circular in shape.

In an example, the trocar 302 may be constructed in two symmetrical halves, each spanning 180 degrees, to facilitate straightforward assembly and disassembly while also allowing the seamless installation of the plurality of sensors on the trocar 302, ensuring precise placement and ease of maintenance. In an example, the trocar 302 comprises an internal groove 324, designed to securely fasten the two symmetrical halves of the trocar body 302 together. In an example, the trocar 302 may include a slot 326 designed to accommodate the wiring for the multiple sensors installed on it. In another example, the trocar 302 may be constructed as a single entity instead of two separate halves, incorporating pre-molded internal grooves and slots for sensor integration and wiring. In an example, the proximal end 310 of the trocar 302 may also comprise a designated space 334 configured for the secure placement of silicon pad. In an example, these silicone pad may serve as a mounting interface that allows the trocar 302 to be firmly held in position on the simulation body, thereby mimicking the interaction between a real trocar and the abdominal wall during laparoscopic procedures.

Thus, the present laparoscopic equipment 300 allows for laparoscopic simulation training, ensuring precision, adaptability, and efficiency in skill development. By integrating the plurality of sensors, the present system provides comprehensive real-time monitoring of critical parameters of the laparoscopic hand instrument, including spatial orientation, depth of insertion, rotational movement, and instrument presence. This sensor-driven approach enables precise data acquisition, allowing trainees to gain hands-on experience with high accuracy, closely mimicking real surgical scenarios. Moreover, the system enhances safety by providing a controlled, simulated environment that eliminates the risks associated with practicing on live patients. Trainees can repeatedly practice complex techniques on the simulated replica organ, gaining confidence and proficiency without real-world consequences.

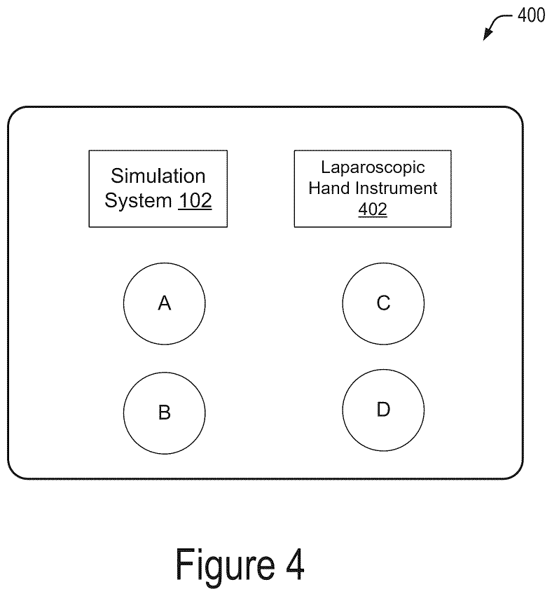

FIG. 4 illustrates a schematic block diagram of a simulator box 400 for accommodating a plurality of trocar 108-1, 108-2, . . . , 108-N and laparoscopic hand instrument 402 for performing laparoscopic surgery on a simulated replica organ, in accordance with an example implementation of the present subject matter.

In an example implementation, the simulator box 400 may comprise a replica abdomen. This replica abdomen may be designed to mimic the anatomical features and tissue properties of a human abdomen, providing a realistic environment for laparoscopic training. In an example, the replica abdomen may comprise a plurality of location A, B, C, and D for placing the trocars 302.

In an example, these locations are strategically positioned within the simulator box 400 to mimic the typical entry points used by the medical professionals in actual laparoscopic procedures. In an example, the multiple placement options allow trainees to practice inserting the trocar 302 at different angles and positions, simulating various surgical scenarios. This arrangement enables trainees 106 to develop proficiency in trocar 302 placement and manipulation, a critical skill in laparoscopic surgery.

In an example, the simulation box 400 may comprise a thermal cautery device and a pulsatile flow system. In an example, the thermal cautery device (not shown), used for dissecting artificial tissues, is securely mounted within the simulator box 400 in a position that allows for safe and convenient access during training. This setup simulates the cautery device's use in real surgical procedures, providing trainees with a realistic experience in handling such equipment.

In an example, the pulsatile flow system (not shown) allows the user 106 for simulating realistic blood flow during surgical procedures, enhancing the training experience. In an example, the simulator box 400 includes a compartment or attachment mechanism for this system, which may include a pump, tank, and connectors. In an example, the pulsatile flow system can simulate various physiological conditions by adjusting flow rates, adding a dynamic element to the overall training process.

In an example implementation, the simulator box 400 may also be equipped with a plurality of cameras 118 installed at various positions within the simulator box. These cameras may be configured to capture multiple images and videos of the user 106 performing the simulation training. In an example, the system 102 may employ advanced image recognition techniques, such as, but not limited to, object tracking, gesture recognition, and motion analysis, to assess the user's performance. For instance, object tracking can monitor the precise movements of the laparoscopic hand instrument 402, gesture recognition can analyze the user's hand positioning, and motion analysis can evaluate the smoothness and accuracy of instrument manipulation. In said example, the collected data may be processed in real time, allowing the system 102 to provide insightful feedback to the user via the user interface 204. This feedback can include performance metrics such as instrument alignment, movement precision, and procedural accuracy, thereby enhancing the effectiveness of the training process and helping trainees refine their laparoscopic skills.

Thus, the simulator box may ensure that trainees are exposed to a wide range of surgical scenarios of the laparoscopy procedures, preparing them for real-life situations with improved proficiency and confidence. The comprehensive design of the simulator box, including multiple trocar placement locations, a replica abdomen, thermal cautery device, and pulsatile flow system, creates a highly realistic training environment. This allows trainees to practice various aspects of laparoscopic surgery, from basic instrument handling to complex procedural techniques. By offering such comprehensive and realistic training experience, the simulator box allows for significant reduction in the learning curve associated with laparoscopic surgery.

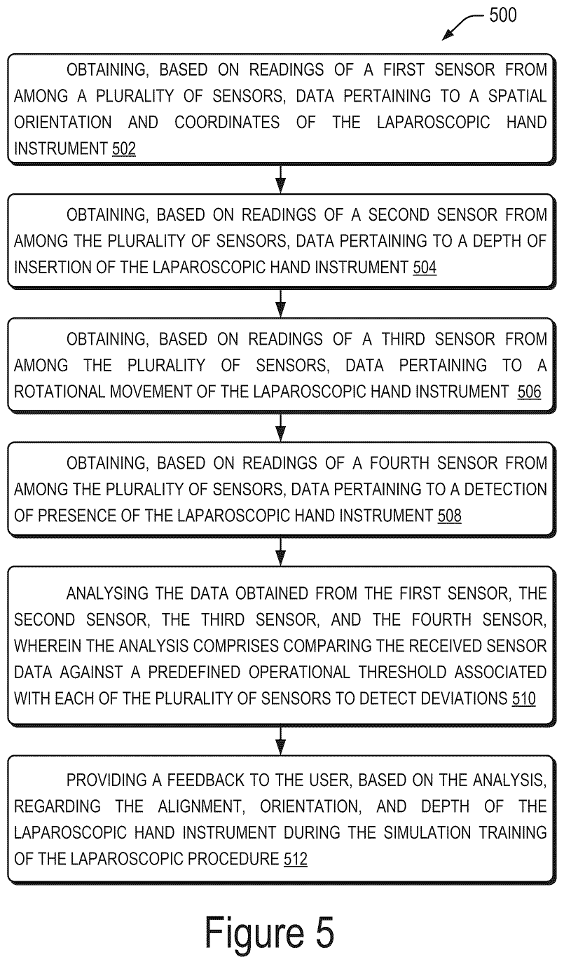

Reference is made to FIG. 5 that illustrates a method for performing a simulation training of the laparoscopic surgery on a simulated box 400, in accordance with an example implementation of the present subject matter.

The order in which the method 500 is described is not intended to be construed as a limitation, and any number of the described method blocks may be combined in any order to implement method 500, or an alternative method. Furthermore, the method 500 may be implemented by processor(s) or computing device(s) through any suitable hardware, non-transitory machine-readable instructions, or combination thereof.

The method 500 for performing a simulation training of the laparoscopic surgery comprises obtaining, from a plurality of sensors, sensor data corresponding to the simulation training of the laparoscopic procedure to provide a feedback to the user on one or more of alignment, orientation, and depth of the laparoscopic hand instrument 400. The steps to provide feedback to the user comprise several steps as illustrated below.

At block 502, the method 500 comprises obtaining, based on readings of a first sensor 110 among a plurality of sensors, data pertaining to a spatial orientation and coordinates of the laparoscopic hand instrument. As explained previously, the first sensor 110 may be an inertial measurement unit (IMU) integrated on the laparoscopic equipment to collect data pertaining to angular velocity, acceleration, and movement of the laparoscopic hand instrument as it interacts with the simulated replica organ. In an example, this data from the first sensor 110 may be referred to as ‘first sensor data’. In an example, the first sensor 110 ensures precise monitoring of the laparoscopic hand instrument's motion, capturing even subtle in trajectory, speed, or stability. In an example, the first sensor 110 works in conjunction with other sensors to provide a tactile feedback to the user 106 operating on the simulated replica organ.

At block 504, the method 500 comprises obtaining, based on readings of a second sensor 112 from among the plurality of sensors, data pertaining to a depth of insertion of the laparoscopic hand instrument. As explained previously, in an example, the second sensor 112 may be a Time-of-Flight (ToF) sensor or an encoder-based sensor, such as, magnetic encoder, to be used for measuring the data pertaining to the depth of insertion of laparoscopic hand instrument by the user 106. The second sensor 112 uses infrared light to calculate the distance between the proximal end of the laparoscopic hand instrument and the targeted tissue boundary target of the simulated replica organ, ensuring precise insertion depth during the procedure in simulation training by the user 106.

At block 506, the method 500 comprises obtaining, based on readings of a third sensor 114 from among the plurality of sensors, data pertaining to a rotational movement of the laparoscopic hand instrument. As explained previously, in an example, the third sensor 114 may be a magnetic encoder that measures the rotational movement of the laparoscopic hand instrument during the simulation training of the laparoscopic procedure. In an example, the third sensor 114 works by utilizing a gear shaft (as shown in FIG. 3) that aligns with an internal hub of the trocar (as shown in FIG. 3). In an example, this internal hub features a specific gear profile. As the laparoscopic hand instrument rotates within the trocar 302, the gear shaft of the third sensor 114 rotates correspondingly. This mechanical interaction allows the third sensor 114 to convert the rotational motion of the laparoscopic hand instrument into quantifiable data.

At block 508, the method 500 comprises obtaining, based on readings of a fourth sensor 116 from among the plurality of sensors, data pertaining to a detection of presence of the laparoscopic hand instrument. As explained previously, in an example, the fourth sensor 116 may be a plurality of proximity sensors, laser sensors, hall effect sensors, etc., embedded around the trocar 302 to serve as a homing device. In an example, the fourth sensor 116 are also configured to detect the presence of one or more surgical tools installed at the proximal end of the laparoscopic hand instrument. The fourth sensor 116 is configured to calculate data pertaining to the detection of the presence of the laparoscopic hand instrument as it approaches the trocar 302 and to ensure precise alignment before insertion by the trainee.

At block 510, the method 500 comprises analysing the data obtained from the first sensor 110, the second sensor 112, the third sensor 114, and the fourth sensor 116. In an example, the analysis comprises comparing the received sensor data against a predefined operational threshold associated with each of the plurality of sensors to detect deviations. As explained previously, in an example, the analysis may comprise receiving sensor data 220 from the plurality of sensors integrated within the trocar 302 of the laparoscopic equipment(s) 104 including the first sensor 110, the second sensor 112, the third sensor 114, and the fourth sensor 116. Further, the received sensor data 220 is compared against a predefined operational threshold associated with each of the plurality of sensors to detect deviations.

In an example, the operational thresholds associated with each of the plurality of sensors may be set by experts in the field of laparoscopic surgery. These thresholds may represent optimal ranges or limits for various parameters such as instrument alignment, orientation, depth of insertion, and rotational movement.

At block 512, the method 500 comprises providing a feedback to the user, based on the analysis, regarding the alignment, orientation, and depth of the laparoscopic hand instrument during the simulation training of the laparoscopic procedure.

By utilizing a plurality of sensors, the method for simulation training of laparoscopic procedures provides a comprehensive and precise approach to surgical skill development. These plurality of sensors capture critical parameters such as spatial orientation, depth of insertion, rotational movement, and instrument presence. This ensures highly accurate monitoring process during the simulation training. Further, by analyzing sensor data against expert-validated operational thresholds, the method ensures objective and standardized performance assessment, helping users identify and rectify errors effectively while also increasing confidence before transitioning to actual surgical procedures.

Although examples for the present disclosure have been described in language specific to structural features and/or methods, it is to be understood that Such examples are not necessarily limited to the specific features or methods described. Rather, the specific features and methods are disclosed and explained as examples of the present description.

I/We claim:

1. A system for simulation training of a laparoscopic procedure, the system comprising:
a plurality of laparoscopic equipment, for performing the simulation training on a simulated replica organ, the laparoscopic equipment comprising:
a plurality of trocars having a funnel-shaped upper portion transitioning into a cylindrical lower portion, wherein each of the plurality of trocar comprising:
an elongated hollow cannula having a proximal end positioned near the simulated replica organ and a distal end extending away from the simulated replica organ;
a sleeve configured to be inserted inside the elongated hollow cannula, the sleeve comprising a plurality of grooves that align with an internal hub located within the upper portion of the trocar;
wherein the internal hub comprises a first gear profile that corresponds to a second gear profile located within the upper portion of the trocar, such that as the sleeve rotates, the second gear profile also rotates;
a laparoscopic hand instrument, which comprises:
an elongated shaft configured to move telescopically and rotate in unison with the sleeve within the hollow cannula of the trocar and insertable into an internal cavity of the simulated replica organ,
wherein the elongated shaft having a proximal end and a distal end, with the proximal end reaching into the internal cavity of the simulated replica organ and configured to accommodate one or more surgical instruments; and
wherein the upper portion of each of the plurality of trocar houses a plurality of sensors, the plurality of sensors includes:
a first sensor configured to measure a spatial orientation and coordinates of the laparoscopic hand instrument during the simulation training of the laparoscopic procedure;
a second sensor configured to measure depth of insertion of the laparoscopic hand instrument during the simulation training of the laparoscopic procedure; and
a third sensor configured to measure rotational movement of the laparoscopic hand instrument during the simulation training of the laparoscopic procedure;
a fourth sensor configured as a homing device to detect presence of the laparoscopic hand instrument during the simulation training of the laparoscopic procedure; and
a processor operatively coupled to the plurality of sensors to provide a feedback during the simulation training of the laparoscopic procedure.

2. The system as claimed in claim 1, wherein the processor is being configured to:
receive data from the plurality of sensors integrated into the trocar of the laparoscopic equipment including the first sensor, the second sensor, the third sensor, and the fourth sensor;
compare the received data against a predefined operational threshold associated with each of the plurality of sensors to detect deviations; and
transmit the processed data to a user interface to provide real-time feedback on laparoscopic instrument alignment, orientation, and depth during the simulation training of the laparoscopic procedure,
wherein the processed data may comprise actionable insights or corrective feedback on alignment, orientation, and depth of using laparoscopic hand instrument on the simulated replica organ.

3. The system as claimed in claim 1, wherein the first sensor is an inertial measurement sensor positioned at a specified location along the upper portion of the trocar.

4. The system as claimed in claim 1, wherein the second sensor is at least one of a time-of-flight sensor, Linear Variable Differential Transformer sensor, magnetic encoder-based sensor positioned at a specified location along the upper portion of the trocar.

5. The system as claimed in claim 1, wherein the third sensor is a non-contact magnetic encoder positioned at the second gear profile in the upper portion of the trocar, measuring the rotation of the laparoscopic hand instrument and converting it into quantifiable data as the sleeve rotates during the rotation of the laparoscopic hand instrument.

6. The system as claimed in claim 1, wherein the fourth sensor is a proximity sensor installed on the tip of the upper portion of the trocar.

7. A method for a simulation training of a laparoscopic procedure for a user using a laparoscopic equipment comprising:
obtaining, from a plurality of sensors, sensor data corresponding to the simulation training of the laparoscopic procedure to provide a feedback to the user on one or more of alignment, orientation, and depth of a laparoscopic hand instrument, wherein providing the feedback to the user comprises:
obtaining, based on reading of a first sensor from among the plurality of sensors, data pertaining to a spatial orientation and coordinates of the laparoscopic hand instrument;
obtaining, based on readings of a second sensor from among the plurality of sensors, data pertaining to a depth of insertion of the laparoscopic hand instrument;
obtaining, based on readings of a third sensor from among the plurality of sensors, data pertaining to a rotational movement of the laparoscopic hand instrument;
obtaining, based on readings of a fourth sensor from among the plurality of sensors, data pertaining to a detection of presence of the laparoscopic hand instrument;
analyzing the data obtained from the first sensor, the second sensor, the third sensor, and the fourth sensor, wherein the analysis comprises comparing the received sensor data against a predefined operational threshold associated with each of the plurality of sensors to detect deviations; and
providing a feedback to the user, based on the analysis, regarding the alignment, orientation, and depth of the laparoscopic hand instrument during the simulation training of the laparoscopic procedure.
